(19) United States
(12) Patent Application Publication
(10) Pub. No.: US 2018/0078525 A1
(43) Pub. Date: Mar. 22, 2018

(54) COMPOSITION FOR GROWTH PROMOTION, CONTAINING COUMARIC ACID AS ACTIVE INGREDIENT

(71) Applicants: Ji Hoon JEONG, Uiwang-si, Gyeonggi-do (KR); Hohyun KIM, Gyeonggi-do (KR); Sooyeon KIM, Uiwang-si, Gyeonggi-do (KR); KOREA MEDICINE RESEARCH INSTITUTE, INC, Uiwang-si, Gyeonggi-do (KR)

(72) Inventors: Hohyun KIM, Uiwang-si (KR); Sooyeon KIM, Uiwang-si (KR); Ji Hoon JEONG, Uiwang-si (KR)

(73) Assignees: NOVATE CO., LTD., Seongnam-si, Gyeonggi-do (KR); Hohyun KIM, Uiwang-si, Gyeonggi-do (KR); Sooyeon KIM, Uiwang-si, Gyeonggi-do (KR)

(21) Appl. No.: 15/539,487

(22) PCT Filed: Apr. 14, 2015
(86) PCT No.: PCT/KR2015/003691

§ 371 Date: Jun. 23, 2017

(30) Foreign Application Priority Data
Jun. 13, 2014 (KR) 10-2014-0071949

(51) Int. Cl. A61K 31/37; A23L 33/105

(52) U.S. Cl. CPC A61K 31/37; A23L 33/105

(57) ABSTRACT

The present invention relates to a growth promotion effect of coumaric acid and, more specifically, to a pharmaceutical composition, a food composition, and an animal feed composition, which are for growth promotion and contain coumaric acid as an active ingredient. The composition containing coumaric acid as an active ingredient, of the present invention, promotes chondrocyte proliferation and growth hormone secretion, thereby finally having an effect of promoting growth, and thus the composition of the present invention is effective in not only growth and skeletal formation of growing children and adolescents, but also height growth therapy by being used alone or in combination with growth hormone therapy.

### TECHNICAL FIELD

This application claims priority to and the benefit of Korean Patent Application No. 10-2014-0071949, filed on Jun. 13, 2014, the disclosure of which is incorporated herein by reference in its entirety.

This invention relates to a growth promoting effect of coumaric acid, and more particularly, to a pharmaceutical composition, a food composition and an animal feed composition, which contain coumaric acid as an active ingredient, for growth promotion.

### BACKGROUND ART

In recent years, due to the effects of westernization on body sizes and the increase in average heights, interests in height growth factors are increasing. When defining growth as a broad concept, it includes not only an increase in height but also an increase in the size and function of organs of the body, and when defining it as a narrow concept, it can seem to mean an increase in height. Such an increase in height is achieved by skeletal metabolism including synthesis of cartilage tissue, growth of skeletal length and extensive proliferation of skeletal tissue due to various factors such as nutrition, growth hormone, etc. Bone tissue is specifically differentiated connective tissue, which consists of various types of cells such as mesenchymal cells, cartilage cells, osteoblasts, osteoclasts, bone marrow cells, etc. While a balance between a level of osteoclast-induced bone resorption and a level of osteoblast-induced osteogenesis is maintained, in the growth phase, an osteoblast-induced ability to form bones reaches its peak and the level of osteogenesis far exceeds the level of bone resorption, resulting in bone growth.

Until now, as a method of promoting growth, administration of a growth hormone preparation, Ilizarov surgery, eating health supplement foods, etc. have been used. Particularly, growth hormone therapies which used to be applied for pathological stunted growth in the past are now being applied to children and adolescents with normal heights in a growing phase, as interest in height development is increasing. However, the administration of a growth hormone preparation shows an excellent effect on people who lack growth hormone, but may cause various side effects including acromegaly, prevalence of growth hormone antibodies, a systemic allergic reaction, and hypothyroidism in most people having normal hormone secretion. Besides, the duration and cost of the growth hormone therapy are excessive, and problems of cancer occurrence and disturbance of other growth factors are found. In addition, the Ilizarov surgery is an operation to extend a leg bone after culling the bone, and is very difficult to be used for normal people due to a patient's pain and a cost. The health supplement foods for promoting growth are generally not scientifically proved.

Therefore, it is necessary to develop food materials which have scientifically-proved growth promotion efficacy and are safe.

### SUMMARY OF INVENTION

### Technical Problem

Therefore, the inventors attempted to develop food materials having an activity of effectively promoting growth without side effects, thereby confirming an effect of increasing a total tibial length in a laboratory animal to which coumaric acid is administered, and thus this invention was completed.

Accordingly, this invention is directed to providing a pharmaceutical composition for promoting growth, which contains coumaric acid as an active ingredient.

This invention is also directed to providing a food composition for promoting growth, which contains coumaric acid as an active ingredient.

This invention is also directed to providing an animal feed composition for promoting growth, which contains coumaric acid as an active ingredient.

### Solution to Problem

In one aspect, this invention provides a pharmaceutical composition for promoting growth, which contains coumaric acid as an active ingredient.

In another aspect, this invention provides a food composition for promoting growth, which contains coumaric acid as an active ingredient.

In still another aspect, this invention provides an animal feed composition for promoting growth, which contains coumaric acid as an active ingredient.

In yet another aspect, this invention provides a use of coumaric acid to prepare a growth promoter, a food composition for promoting growth, and animal feed for promoting growth.

In yet another aspect, this invention provides a method of promoting growth, which includes administering an effective amount of coumaric acid to an individual in need of growth.

Hereinafter, this invention will be described in detail.

This invention provides a composition for promoting growth, which contains coumaric acid an active ingredient.

Coumaric acid is any one or more selected from the group consisting of o-coumaric acid, m-coumaric acid, and p-coumaric acid.

Coumaric acid is also called hydroxycinnamic acid, and has three geometric isomers (o-coumaric acid, m-coumaric acid, and p-coumaric acid). Any type of isomer of coumaric acid is used without limitation.

Coumaric acid is contained in plants such as peanuts, tomatoes, carrots and garlic, and also found in wine, vinegar and grains.

Coumaric acid may be obtained by extraction from plants such as peanuts, tomatoes, carrots, garlic, or Sasa quelpaertensis Nakai, or may be directly purchased from Sigma-Aldrich (St. Louis, Mo., USA).

Coumaric acid has been reported to have an effect of reducing a risk of stomach cancer when eaten due to an antioxidant property. In addition, in numerous studies, coumaric acid has been disclosed as an inhibitor of melanin production, and has been reported to have an inhibitory effect on bacterial growth. For this reason, coumaric acid is being studied as a natural whitening substance. However, a growth promotion effect of of coumaric acid has not been reported.

The “growth” used herein refers to an increase in the size and function of organs in the body, preferably, to an increase in the size, thickness, density, length and function of tissue of a proliferative bone, and an increase in growth hormone, and further preferably, to the longitudinal growth of a bone.

The composition of this invention contains coumaric acid an active ingredient, and is effective in the longitudinal growth.

This is well disclosed in the specification.

In an exemplary embodiment of this invention, coumaric acid was administered to rats for 10 days, and a change in total tibial length was observed. As a result, it was confirmed that the total tibial length was increased similar to that of the growth hormone-administered group (FIG. 2).

In another exemplary embodiment of this invention, coumaric acid was administered to rats for 10 days, and heights of the proliferative zone and the hypertrophic zone of a growth plate. As a result, it was confirmed that the heights of the proliferative zone and the hypertrophic zone of the growth plate were increased similar to the growth hormone-administered group (FIG. 3).

In still another exemplary embodiment of this invention, it was confirmed that, when coumaric acid was administered to rats, the proliferation of cartilage cells was increased, compared to a control group (FIG. 4).

In yet another exemplary embodiment of this invention, it was confirmed that, when coumaric acid was administered to rats, the growth hormone in blood was increased (FIG. 5), and the level of IGF-1 protein, which promotes bone growth by stimulating secretion of the growth hormone, was increased (FIG. 6).

Therefore, this invention provides a pharmaceutical composition for promoting growth, which contains coumaric acid as an active ingredient, a use of coumaric acid to prepare a growth promoter, and a method of promoting growth, which includes administering an effective amount of coumaric acid to an individual in need of growth.

The “effective amount” used herein refers to an amount exhibiting a growth promoting effect when a material is administered to a subject, and the “individual” used herein may be an animal, preferably, a mammal including particularly a human, and the individual may be a subject in need of growth promotion.

The “growth” used herein refers to an increase in the size and function of organs in the body, and to an increase in the size, thickness, density, length and function of tissue of a proliferative bone, and an increase in growth hormone, and further preferably, to the longitudinal growth of a bone.

The pharmaceutical composition according to this invention may contain the coumaric acid of this invention alone, or further contain one or more of a pharmaceutically acceptable carrier, an excipient and a diluent. Here, the “pharmaceutically acceptable” used herein refers to a non-toxic composition which does not inhibit the action of an active ingredient and does not normally cause allergic reactions such as gastrointestinal disorder, and dizziness or similar reactions thereto when a material is acceptable physiologically and administered to a human.

As a pharmaceutically acceptable carrier, for example, a carrier for oral administration or parenteral administration may also be used. The carrier for oral administration may include lactose, starch, a cellulose derivative, magnesium stearate, stearic acid, etc. Various drug delivery systems used for oral administration of a peptide preparation may also be included. In addition, the carrier for parenteral administration may include water, suitable oil, a saline solution, aqueous glucose, glycol, etc., and further include a stabilizing agent and a preservative. As a suitable stabilizing agent, an antioxidant such as sodium hydrogen sulfite, sodium sulfite or ascorbic acid is used. As a suitable preservative, benzalkonium chloride, methyl- or propyl-paraben or chlorobutanol is used. The pharmaceutical composition of this invention may further include a lubricant, a wetting agent, a sweetening agent, a flavor, an emulsifier, or a suspending agent, in addition to the above-described components. Other pharmaceutically acceptable carriers and agents may be referred to items which are disclosed in the following literature (Remington's Pharmaceutical Sciences, 19th ed., Mack Publishing Company, Easton, PA, 1995).

The composition of this invention may be administered to mammals including a human by any method. For example, the composition may be administered orally or parenterally. The parenteral administration method may be, but is not limited to, intravenous, intramuscular, intraarterial, intramedullar, intrathecal, intracardiac, transcutaneous, subcutaneous, intraperitoneal, intranasal, enteral, topical, sublingual or rectal administration.

The pharmaceutical composition of this invention may be formulated in preparations for oral or parenteral administration according to an administration route as described above.

For the preparations for oral administration, the composition of this invention may be formulated in the form of powders, granules, tablets, pills, sugar-coated tablets, capsules, liquids, gel, syrup, slurry or suspensions using a method known in the art. For example, the preparations for oral administration may be prepared as tablets or sugar-coated tablets by mixing active ingredients with solid excipients, grinding the resulting mixture, adding suitable additives, and then processing it into a granular mixture. Examples of suitable excipients may include saccharides including lactose, dextrose, sucrose, sorbitol, mannitol, xylitol, erythritol and maltitol, starches including corn starch, wheat starch, rice starch and potato starch, celluloses including cellulose, methyl cellulose, sodium carboxymethyl-cellulose and hydroxypropylmethyl-cellulose, and fillers such as gelatin and polyvinylpyrrolidone. In addition, a disintegrant such as crosslinked polyvinylpyrrolidone, agar, alginic acid or sodium alginate may be optionally added. Furthermore, the pharmaceutical composition of this invention may further include an anti-aggregating agent, a lubricant, a wetting agent, a flavor, an emulsifier, a preservative, etc.

For preparations for parenteral administration, the composition of this invention may be formulated as injections, creams, lotions, ointments, oil, emulsions, gel, aerosols or nasal inhalants according to a method known in the art. These formulations are disclosed in the following literature (Remington's Pharmaceutical Science, 19th ed., Mack Publishing Company, Easton, PA, 1995), which is the formulary generally known in all fields of pharmaceutical chemistry.

The total effective amount of the composition of this invention may be administered to a subject as a single dose, or administered as multiple doses according to a fractionated treatment protocol. The pharmaceutical composition of this invention may contain various contents of the active ingredient according to the severity of a disease. A preferable dose of the pharmaceutical composition of this invention may be approximately 0.01 to 10,000 mg, and most preferably 0.1 to 1000 mg per kilogram of the body weight of a subject daily. However, the effective dose of the pharmaceutical composition for a subject is determined by considering various parameters such as a subject's age, body weight, health condition, sex, severity of a disease, diet and excretion ratio, as well as a formulation method, an administration route and a treatment cycle. In consideration of these parameters, a suitable effective dose of the composition of this invention may be determined by those of ordinary skill in the art. There are no particular limits to a formulation, administration route and administration method of the pharmaceutical composition of this invention as long as the composition exhibits the effects of this invention.

This invention provides a food composition for promoting growth, which contains coumaric acid as an active ingredient and a use of coumaric acid to prepare a food composition for promoting growth.

The food composition of this invention includes all types of functional foods, nutritional supplements, health foods and food additives. The above type of food composition may be prepared in various forms according to a method conventionally known in the art.

For example, as health food, the coumaric acid of this invention may be taken in the form of tea, juice or a drink, or ingested in the form of granules, capsules and powder. In addition, the coumaric acid of this invention may be mixed with a material or active ingredient, which has been known to have a growth promoting effect to be prepared as a composition. For example, the food composition of this invention may further contain, in addition to coumaric acid or the coumaric acid component, trace amounts of minerals, vitamins, lipids, saccharides and known components having a growth promoting activity. The minerals may include nutrient components necessary for a growth phase, such as calcium, iron, etc., and the vitamins may include vitamin C, vitamin E, vitamin B1, vitamin B2, vitamin B6, etc. The lipids may include alkoxyglycerol or lecithin, and the saccharides may include fructooligosaccharides.

In addition, the functional food may be prepared by adding the coumaric acid of this invention to beverages (including alcoholic beverages), fruits and processed foods thereof (e.g., canned fruit, bottled food, jam, marmalade, etc.), fish, meat and processed foods thereof (e.g., ham, sausage, corned beef, etc.), breads and noodles (e.g., Udon, buckwheat noodles, ramen, spaghetti, macaroni, etc.), fruit juice, all types of drinks, cookies, taffy, dairy products (e.g., butter, cheese, etc.), edible vegetable oil, margarine, vegetable protein, retort food, frozen food, or various seasonings (e.g., soybean paste, soy sauce, other kinds of sauces, etc.).

In addition, to be used as a food additive, the coumaric acid of this invention may be used may be prepared as powder or a concentrate.

A preferable content of the coumaric acid in the food composition of this invention may be 0.01 to 90 wt %, and preferably 0.1 to 50 wt % with respect to a total weight of the food composition.

This invention provides an animal feed composition for promoting growth, which contains coumaric acid as an active ingredient, and a use of coumaric acid to prepare animal feed for promoting growth.

The feed composition according to this invention may be prepared as fermented feed, compound feed, pellet-type feed or silage-type feed. The fermented feed may include the coumaric acid of this invention, and may be prepared by further adding various microorganisms or enzymes and fermenting the organic material. The compound feed may be prepared by mixing various types of regular feed with the coumaric acid of this invention. The pellet-type feed may be prepared by formulating the fermented feed or compound feed using a pellet machine. The silage-type feed may be prepared by mixing soilage with the coumaric acid of this invention and fermenting the resulting mixture using lactic acid bacteria.

### Advantageous Effects of Invention

Compositions containing coumaric acid as an active ingredient according to this invention promote the proliferation of cartilage cells and growth hormone secretion, resulting in growth promotion, and therefore they are effective for the growth and skeletal formation of children and adolescents in a growing phase, and also effective for height growth treatment when used alone or in combination with growth hormone therapy.

### BRIEF DESCRIPTION OF DRAWINGS

FIG. 1 is a diagram illustrating changes in body weight of rats in administration groups for 10 days.

FIG. 2 is a graph illustrating total tibial length (Data represent mean (n=5), *p<0.05).

FIG. 3A is a graph illustrating the height of the growth plate of rats of each administration group, and FIGS. 3B and 4C are graphs illustrating the heights of a proliferative zone and a hypertrophic zone (A: 20× magnification, *p<0.05 and ***p<0.001).

FIG. 4A is an image of the proximal tibia growth plate, stained with BrdU, in rats of each administration group, and FIG. 5B is a graph illustrating the ratio of BrdU-labeled cartilage cells in rats of each administration group (A: 40× magnification, *p<0.05).

FIG. 5 is a graph illustrating the growth hormone levels in the blood of rats in each administration group (Data represent mean (n=5), *p<0.05).

FIG. 6A is an image of the proximal tibia growth plate, stained with BrdU, in rats of each administration group, and FIG. 6B is a graph illustrating the levels of IGF-1 in the blood of rats in each administration group (A: 20× magnification, B: **p<0.01 and ***p<0.001).

### DETAILED DESCRIPTION OF EMBODIMENT

Hereinafter, this invention will be described in detail.

However, the following examples are merely provided to exemplify this invention, not to limit the scope of this invention.

### EXPERIMENTAL EXAMPLE 1

Assessment of Growth Promoting Effect of Coumaric Acid Using Animal Models

<1-1> Administration of Coumaric Acid

Three-week-old male Sprague-Dawley rats were purchased and acclimated for three days. A breeding environment was altered to a 24-hour pattern, a light/dark cycle was maintained at 12-hour intervals, and antibiotic-free normal solid feed was used. In a control group, saline was injected. In a positive control group, Eutropin Injection (LG Bioscience Korea) was subcutaneously injected at a dose of 20 μg/kg once a day. The coumaric acid of this invention was administered to the oral cavity of all rats of a coumaric acid group, once daily. The drug administered to each group is as follows:

Control: normal saline, 1 ml

Positive Control: growth hormone, 20 μg/kg

Coumaric acid: 100 mg/kg

After feeding and administering the coumaric acid to the rats for 10 days, autopsies were carried out to confirm the following content.

<1-2> Changes in the Body Weight of Laboratory Animals

Body weights before feeding and body weights after feeding for 10 days were measured for each group of Experimental Example <1-1>. The measurement was carried out twice a week. The measured values were statistically analyzed to calculate mean±standard deviation.

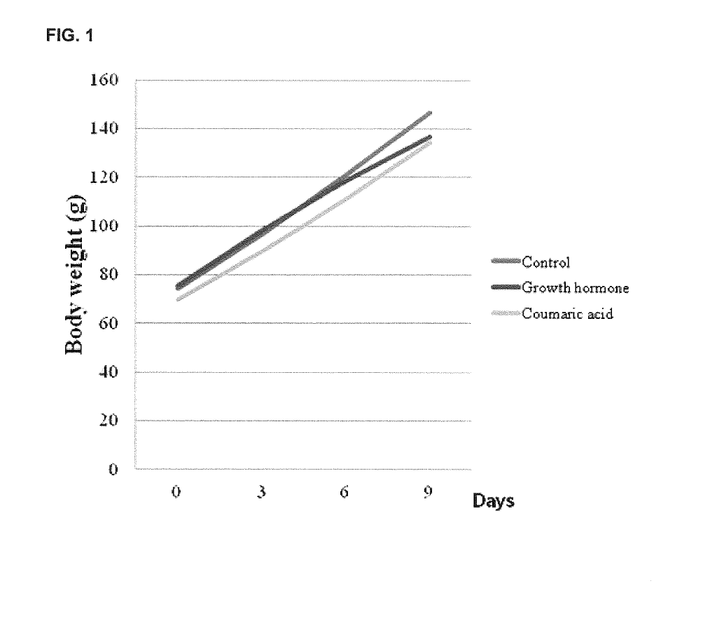

As shown in FIG. 1, the 10-day observation showed that there were almost no difference between the body weights in all of the control group, the experimental group, and growth hormone-administered group. It shows that there is no close correlation between the body weight of a mouse and the effect of coumaric acid administration.

<1-3> Measurement of Changes in the Total Tibial Length of Laboratory Animals

To assess the effect of coumaric acid on growth promotion, total tibial length of rats were measured. In Experimental Example <1-1>, after the 10-day administration was completed, right and left tibias (shinbones) were extracted from the control groups and the coumaric acid-fed experimental group according to this invention, all the muscles, fat and ligaments attached to bone tissue were removed, and then the resulting specimen was stored in 70% alcohol, followed by radiographic analysis using an X-ray imaging instrument (OM-FORTE-10121, DK Medical Systems). For the analysis, 55 kV, 320 A X-ray sources were used. The total tibial length was measured by X-ray radiography.

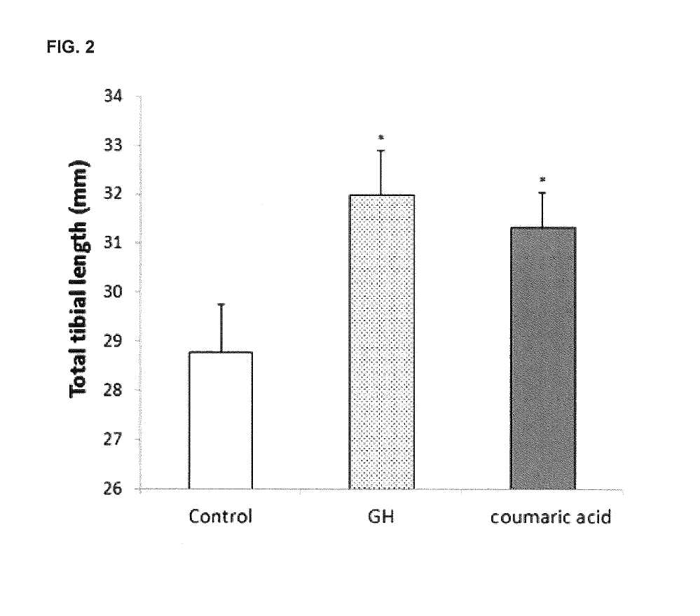

As shown in FIG. 2, it can be seen that the total tibia length of the growth hormone-administered group was greater than the control group, and the total tibial length of the coumaric acid-administered group was also greater than the control group.

<1-4> Analysis of Growth Plate in Laboratory Animals

To assess the growth effect of coumaric acid, the growth plate of each rat was analyzed. In Experimental Example <1-1>, after the 10-day administration was completed, right and left tibias (shinbones) were extracted from the experimental group and the control groups according to this invention, all the muscles, fat and ligaments attached to bone tissue were removed, and then the resulting specimen was stored in 70% alcohol, demineralized, and then dehydrated. Afterward, the resulting specimen was washed with xylene, and then put into a paraffin block. Subsequently, the specimen was cut to a thickness of 4 μm, and then the heights of the proliferative zone and the hypertrophic zone in the growth plate were measured using a BX50 microscope (Olympus).

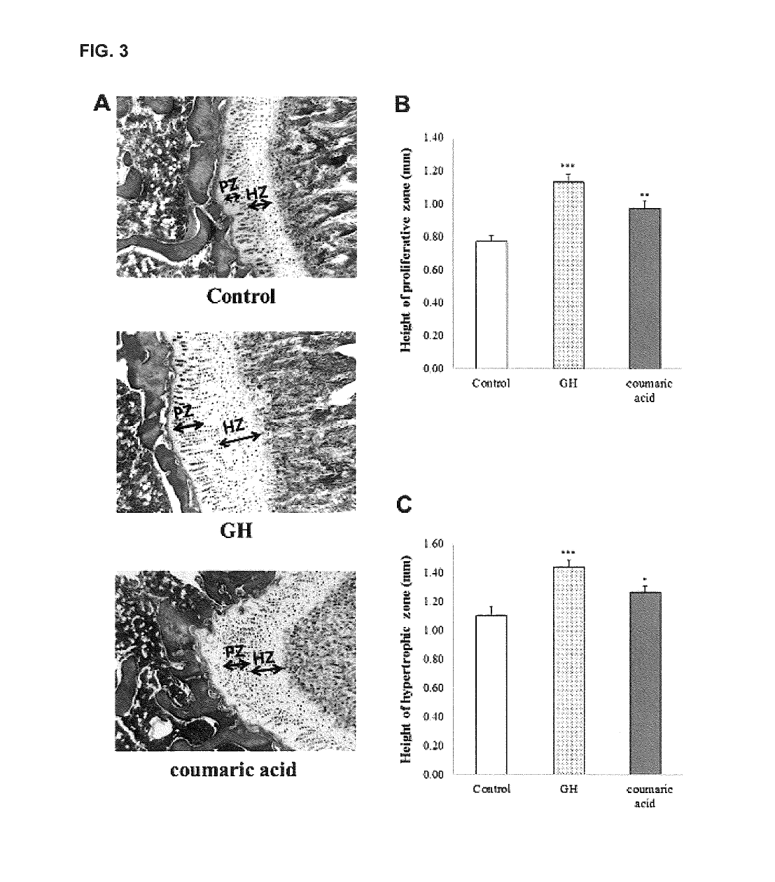

As shown in FIG. 3, on average, the height of the proliferative zone of the control group was 0.77 mm, and the height of the proliferative zone of the growth hormone-administered group was 1.13 mm. In addition, the height of the proliferative zone of the coumaric acid-administered group was measured at 0.98 mm. It was confirmed that both of the growth hormone-administered group and the coumaric acid-administered group showed significant increases in the height of the proliferative zone, compared to the control group, and particularly, the increase in the height of the proliferative zone of the coumaric acid-administered group was similar to that of the growth hormone-administered group. It was also confirmed that the height of the hypertrophic zone was 1.44 mm in the growth hormone-administered group, and 1.27 mm in the coumaric acid-administered group, both of which were greater than that of the control group (1.10 mm).

<1-5> Observation of Proliferation of Cartilage Cells in Proximal Tibia in Laboratory Animals

To confirm whether the effect of coumaric acid on bone growth is caused by the proliferation of cartilage cells, cartilage cells were observed. In Experimental Example <1-1>, after the 10-day administration was completed, rats of the experimental group and the control groups according to this invention were intraperitoneally administered with a dose of 30 mg/kg of 5-bromo-2′-deoxyuridine (BrdU) on the day of the experiment. Two hours later, autopsies were carried out to extract legs, and then the legs were stored in 4% PFA for 24 hours. Following embedding and sectioning, cartilage cells which were stained using a BrdU staining kit (Invitrogen, Carlsbad, Calif., USA) were observed.

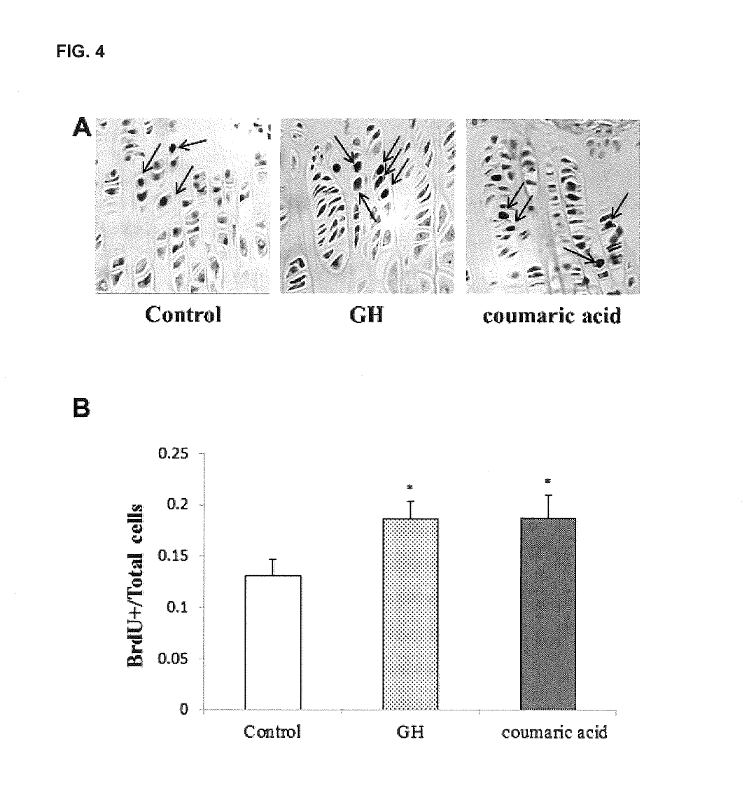

As shown in FIG. 4, a ratio of the BrdU-labeled cells with respect to total cells was 0.13% in the control group, 0.18% in the growth hormone-administered group, and 0.18% in the coumaric acid-administered group. In other words, the ratios of the BrdU-labeled cartilage cells were increased in both of the growth hormone-administered group and the coumaric acid-administered group, compared to the control group.

<1-6> Measurement of Growth Hormone Levels in Blood of Laboratory Animals

To confirm whether coumaric acid affects the secretion level of the growth hormone, a growth hormone level in the blood of each rat was measured. In Experimental Example <1-1>, after the 10-day administration was completed, blood was collected from the inferior vena cava of the rats in the experimental group and the control groups according to this invention on the day of the experiment, and then centrifuged to obtain serum. The measurement of the growth hormone levels were carried out using a Growth Hormone Rat ELISA Kit (Life Technologies, KRC5311).

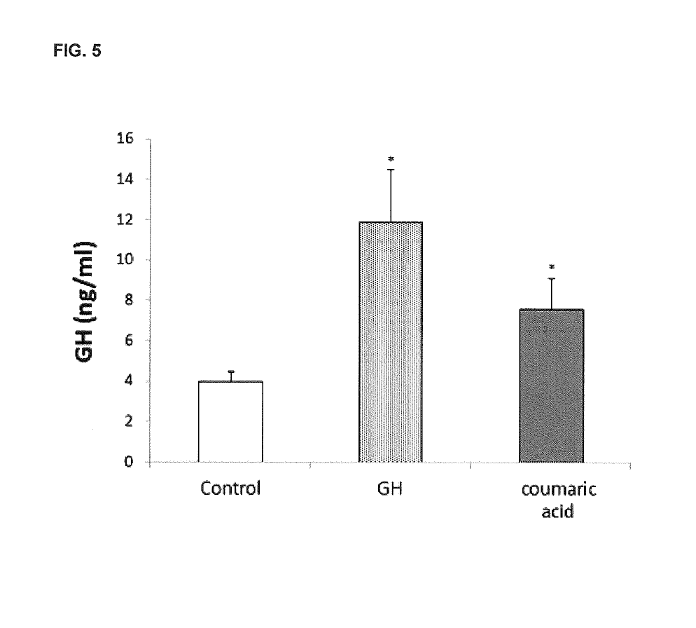

As shown in FIG. 5, the growth hormone levels in blood were 3.97 ng/ml in the control group, 11.89 ng/ml in the growth hormone-administered group, and 7.62 ng/ml in the coumaric acid-administered group, which showed that the growth hormone levels were significantly increased in both of the growth hormone-administered group and the coumaric acid-administered group, compared to the control group.

<1-7> Measurement of IGF-1 Protein Levels in Blood of Laboratory Animals

To confirm whether coumaric acid affects the secretion levels of IGF-1, which is a protein that stimulates growth hormone secretion, IGF-1 protein levels were measured. In Experimental Example <1-1>, after the 10-day administration was completed, the rats of the experimental group and the control groups according to this invention were anesthetized on the day of the experiment, and then blood was extracted therefrom using a syringe and centrifuged to obtain serum. The measurement of the IGF-1 levels was carried out using an IGF1 ELISA Kit (Abcam, ab100695).

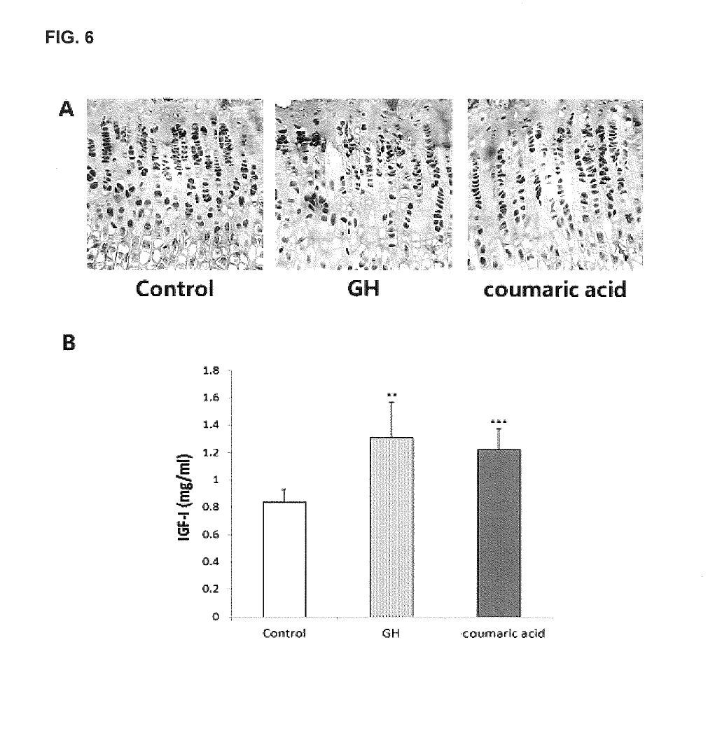

As shown in FIG. 6, the IGF-1 protein levels were 0.84 mg/kg in the control group, 1.31 mg/kg in the growth hormone-administered group, and 1.23 mg/kg in the coumaric acid-administered group, which showed that the IGF-1 protein levels were significantly increased in both of the growth hormone-administered group and the coumaric acid-administered group, compared to the control group.

### INDUSTRIAL AVAILABILITY

As described above, this invention relates to a growth promoting effect of coumaric acid, and more particularly, to a pharmaceutical composition, a food composition and an animal feed composition, which contain coumaric acid as an active ingredient, for promoting growth.

The compositions containing coumaric acid an active ingredient according to this invention have an effect of promoting the proliferation of cartilage cells and growth hormone secretion, resulting in the promotion of growth. Therefore, the compositions of this invention are effective for the growth and skeletal formation of children and adolescents in a growing phase, and also effective in height growth treatment when used alone or in combination with growth hormone therapy, and thus have high industrial availability.

1. A method for pormoting growth, the method comprising administering to a subject in need thereof an effective amount of coumaric acid as an active ingredient.

2-3. (canceled)

4. The method according to claim 1, wherein the coumaric acid is any one or more selected from the group consisting of o-coumaric acid, m-coumaric acid, and p-coumaric acid.

5. The method according to claim 1, wherein the growth is bone growth.

6-9. (canceled)
